(19) United States
(12) Patent Application Publication
(10) Pub. No.: US 2014/0350418 A1
(43) Pub. Date: Nov. 27, 2014

(54) BLOOD PRESSURE KIOSK FOR USE WITH LEFT OR RIGHT ARM

(71) Applicant: PHARMA-SMART INTERNATION, INC., ROCHESTER, NY (US)

(72) Inventor: DREW LANDON KNOPFEL, FAIRPORT, NY (US)

(73) Assignee: PHARMA-SMART INTERNATION, INC., ROCHESTER, NY (US)

(21) Appl. No.: 13/898,787

(22) Filed: May 21, 2013

(51) Int. Cl. A61B 5/021; A61B 5/022

(52) U.S. Cl. CPC A61B 5/02141; A61B 5/022
USPC 600/490

(57) ABSTRACT

A blood pressure kiosk is provided which allows a user to place his or her left or right arm into the blood pressure cuff for measurement. The kiosk includes either a moving support arm, or a moving seat. The kiosk can also include a moving display so that the user is able to view information on the display from either seat position.

### FIELD OF THE INVENTION

The present invention relates to blood pressure measuring kiosks. More particularly, the invention relates to a blood pressure measuring kiosk which is adapted to facilitate the taking of a blood pressure measurement from either the left arm or the right arm of a user of the kiosk.

### BACKGROUND OF THE INVENTION

Blood pressure kiosks are known. Various retail stores, such as pharmacies, grocery stores, and other retailers provide blood pressure measurement kiosks for use by their customers to measure their blood pressure. These blood pressure kiosks are designed such that a user may place his arm into a blood pressure cuff, and the machine automatically measures the user's blood pressure. The blood pressure reading is typically shown on a display. Some of these kiosks offer advanced features such as allowing the use of smartcards to activate the kiosk and to store blood pressure data over time.

To date, all blood pressure kiosks have been designed to measure the blood of the user from only one of the left or right arm, and typically from the left arm only. However, users of the blood pressure measurement kiosk may have a temporary or permanent disability which prevents the user from placing one of his arms through the measurement cuff. For example, the user may have his left arm in a cast or sling due to an injury, or the user may have had one arm amputated.

A blood pressure measurement kiosk is needed which is capable of taking the blood pressure from the user from either the right or left arm.

### BRIEF SUMMARY OF THE INVENTION

The present invention includes a blood pressure kiosk for measuring the blood pressure of a user. The kiosk includes a base with a control unit mounted on the base. A display is mounted on the base for displaying information, such as the status of the test and the results. A seat is positioned near the base. A support arm supports a blood pressure cuff. The blood pressure cuff could be of the type found in U.S. Pat. No. 7,427,268. In one embodiment, the support arm has two positions, a first position in which the support arm is near the right side of the base and a second position in which the support arm is near the left side of the base. The support arm moves between these two positions by either sliding or pivoting. When the user is sitting on the seat, the user may put his left arm through the blood pressure cuff when the support arm is in the first position and when the user is sitting on the seat, the user may put his right arm through the blood pressure cuff when the support arm is in the second position.

In another embodiment, the seat has two positions, a first position in which the seat is primarily between the left side of the base and right side of the kiosk base, and a second position in which the seat is laterally displaced with respect to the base. The seat may be displaced by sliding it laterally, or pivoting it about a vertical axis. The display may also have two positions. In one embodiment, the display slides between the two positions. In another embodiment, the display is mounted on moving arms which allow the display to move between the two positions. In yet another embodiment, the display rotates about a pivot point between the two positions.

### BRIEF DESCRIPTION OF THE SEVERAL VIEWS OF THE DRAWINGS

The foregoing and other objects, features, and advantages of the invention will be apparent from the following drawings and more particular description of the preferred embodiments of the invention.

FIG. 1 is a perspective view of the present invention;

FIG. 2 is a front elevation view of the invention of FIG. 1;

FIG. 3 is a partial-cutaway perspective view of the invention of FIG. 1;

FIG. 4 is a perspective view of the invention of FIG. 1 showing a rotatable cuff;

FIG. 5 is a perspective view of an embodiment of the present invention showing a pivoting support arm;

FIG. 6 is a perspective view of an embodiment of the present invention showing a sliding support arm;

FIG. 7 is a perspective view of another embodiment of the present invention;

FIG. 8 is a front elevation view of the embodiment of FIG. 7;

FIG. 9 is a front elevation view of the embodiment of FIG. 7 showing the seat in an alternative position;

FIG. 10 is a perspective view of another embodiment of the present invention;

FIG. 11 is a perspective view of another embodiment of the present invention; and

FIG. 12 is a perspective view of another embodiment of the present invention.

### DETAILED DESCRIPTION OF THE INVENTION

Throughout the following description, specific details are set forth in order to provide a more thorough understanding of the invention. However, the invention may be practiced without these particulars. In other instances, well known elements have not been shown or described in detail to avoid unnecessarily obscuring the disclosure. Accordingly, the specification and drawings are to be regarded as illustrative rather than restrictive. It is to be further noted that the drawings are not to scale.

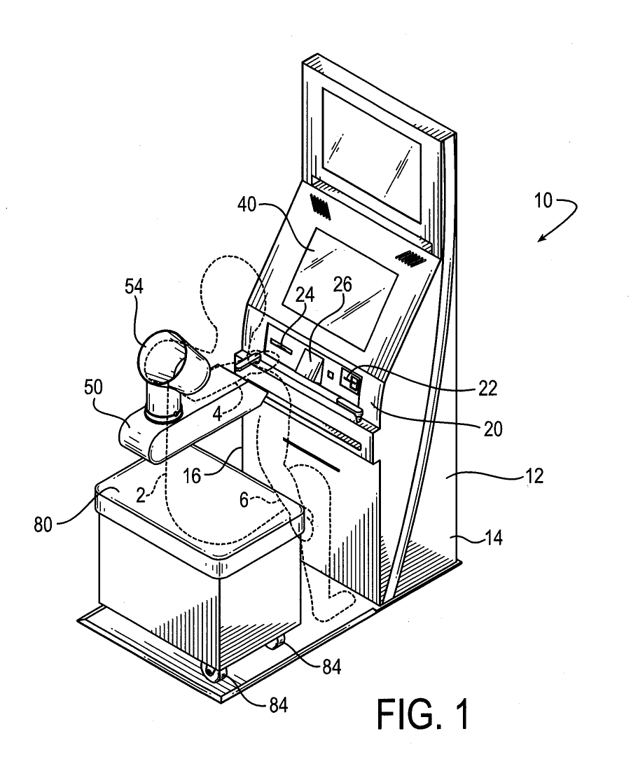

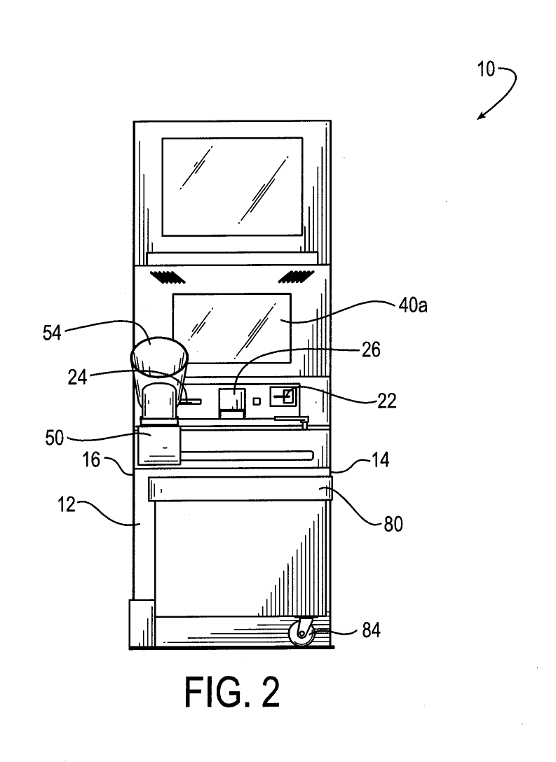

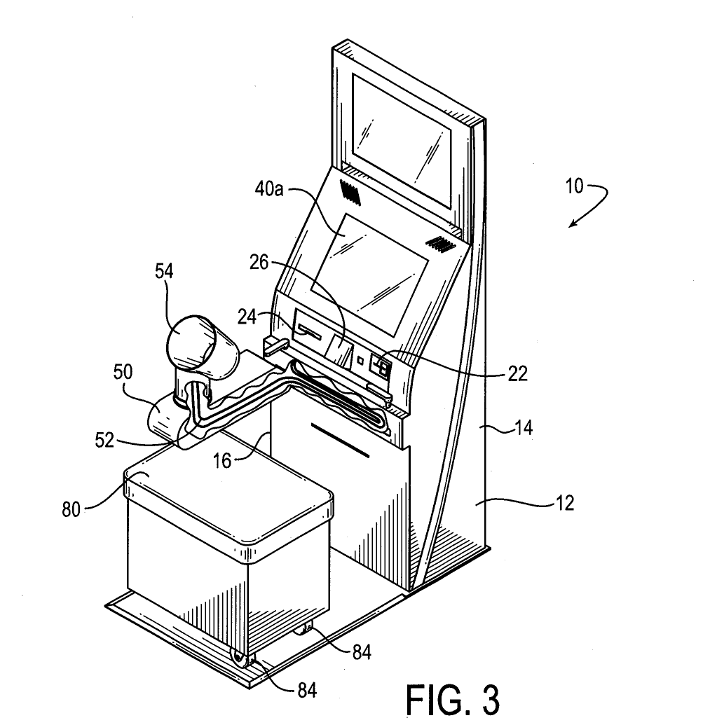

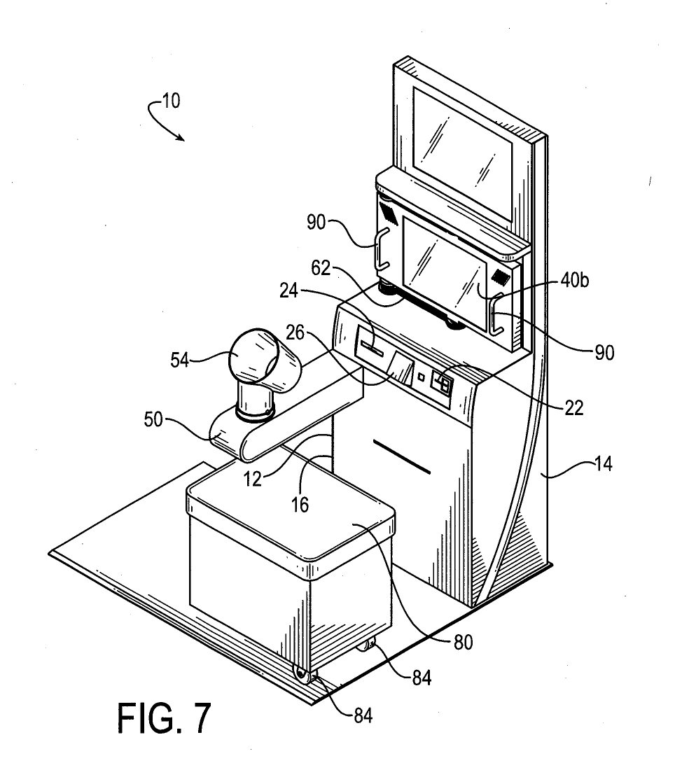

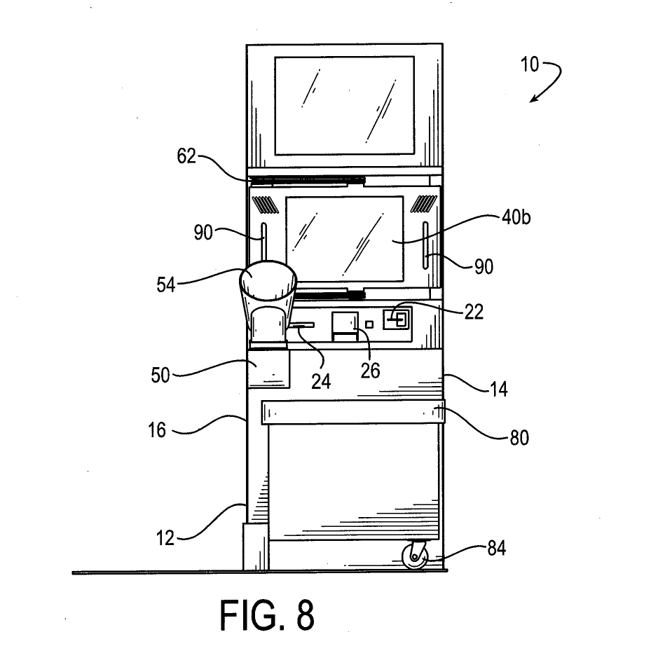

FIGS. 1 through 12 illustrate the invention, with FIGS. 1 through 3 best illustrating the main components of the invention. The blood pressure kiosk 10 includes a base 12, a left side 14, a right side 16, control area 20 display 40a (FIG. 1-6) or 40b (FIGS. 7-12), support arm 50 and seat 80, with wheels 84. The base 12 supports the kiosk 10 and can be made in a variety of shapes. The base houses the electronic and pneumatic controls, as is known in the art. The control area 20 includes card slot 22 for inserting data cards (not shown) into the card slot 22, and includes a barcode reader 24 for reading barcodes on user identification cards. A shroud 26 covers a paper slot (not shown) for preventing a user 2 from removing a printed transaction record before the printing has been completed. The user 2 is shown with his left arm 4 through the blood pressure cuff 54 and his right arm 6 not in the blood pressure cuff 54. As shown in the cutaway portion of FIG. 3, the support arm 50 includes pneumatic and electrical cabling 52 between the blood pressure cuff 54 and a central processing unit (not shown) within the base 12.

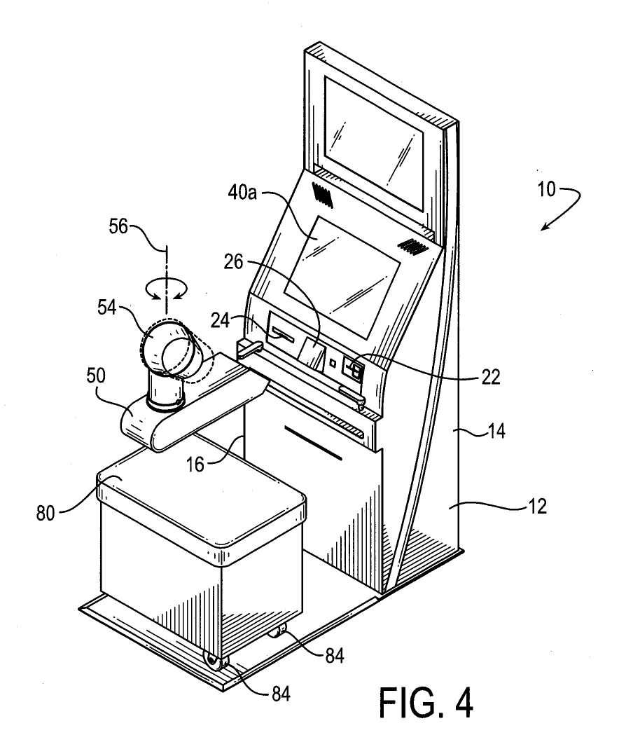

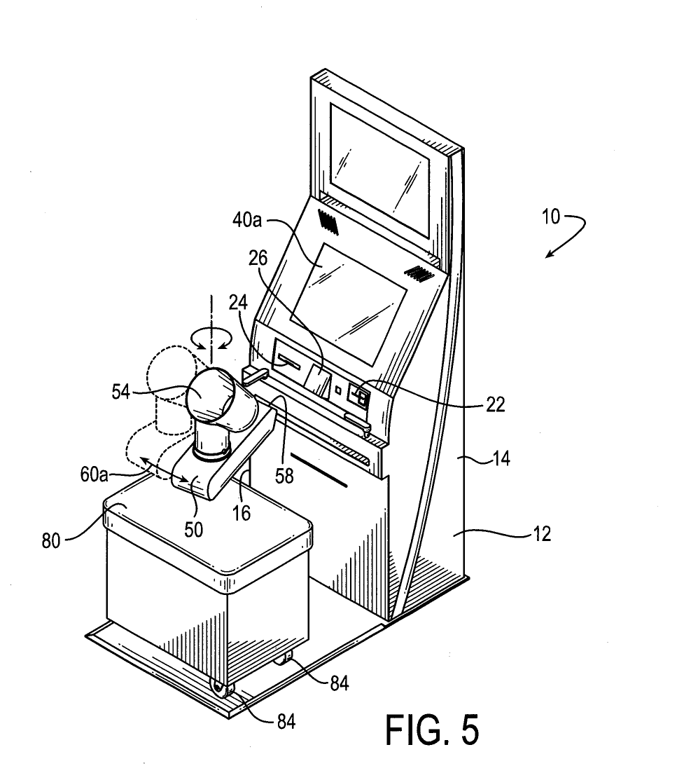

In one embodiment, as shown in FIGS. 4 and 5, the blood pressure cuff 54 rotates about a vertical axis 56. As shown in FIG. 5, the support arm 50 is capable of rotation about pivot point 58 in the direction of arrow 60a. This allows a user (shown in FIG. 1) to insert either a left arm 4 or right arm 6 into the blood pressure cuff 54. By allowing the blood pressure cuff 54 to rotate, the blood pressure cuff 54 can be aligned with the arm of the user 2 (not shown in FIGS. 4 and 5). Also, the support arm 50 can be rotated out of the way to facilitate access to the seat 80.

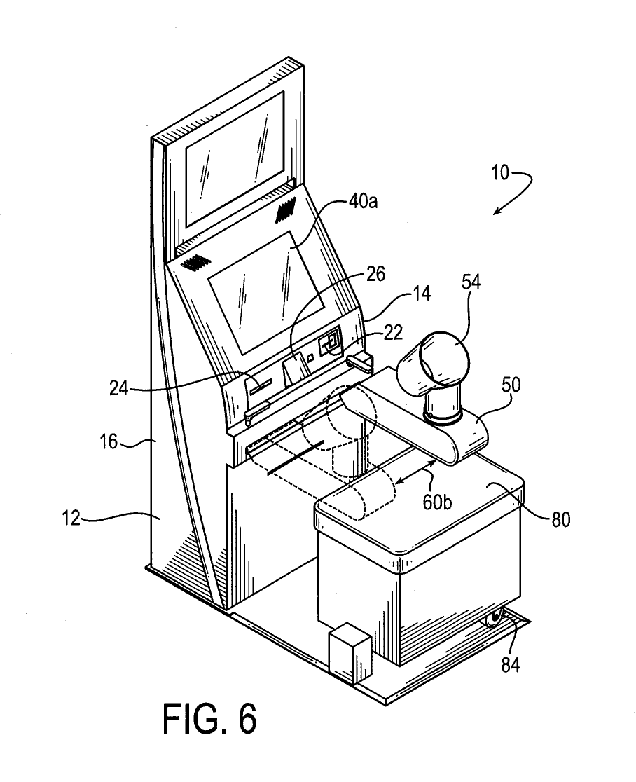

In another embodiment, as shown in FIG. 6, the support arm 50 is capable of sliding in the direction of arrow 60b. This allows a user 2(not shown in FIG. 6) to insert either a left arm 4 or right arm 6 into the blood pressure cuff 54. Both the sliding arm feature of FIG. 6 and the rotating arm feature of FIGS. 4 and 5 can be combined into a single embodiment.

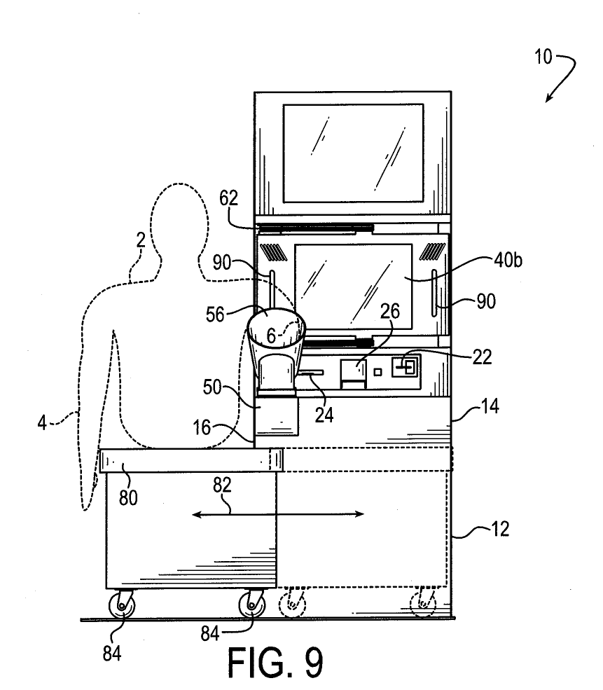

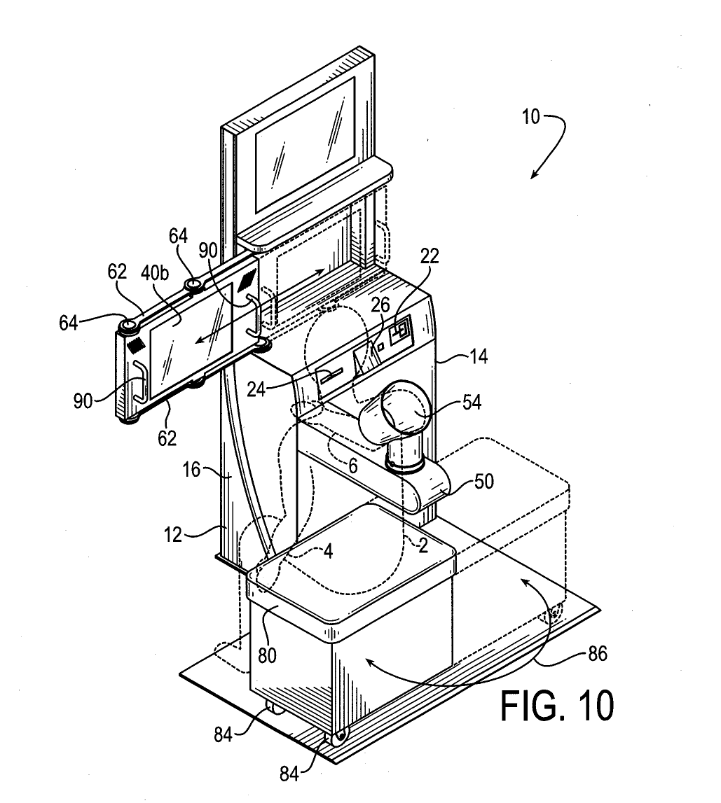

In another embodiment, as shown in FIGS. 7 through 9, the seat 80 is adapted to move in the direction of arrow 82 (FIG. 9). Wheels 84 facilitate movement, but other methods of lateral movement are possible. In another embodiment, as shown in FIG. 10, the seat 80 is adapted for rotational movement in the direction of arrow 86.

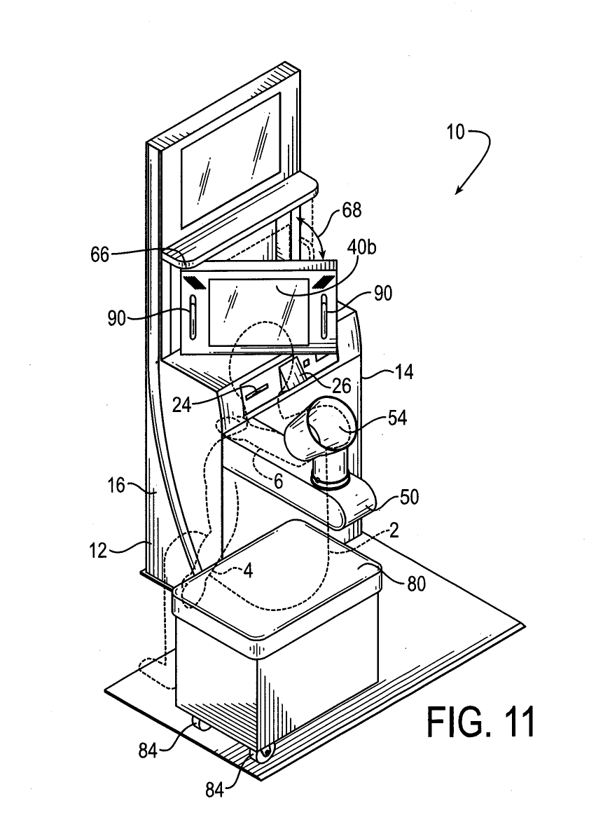

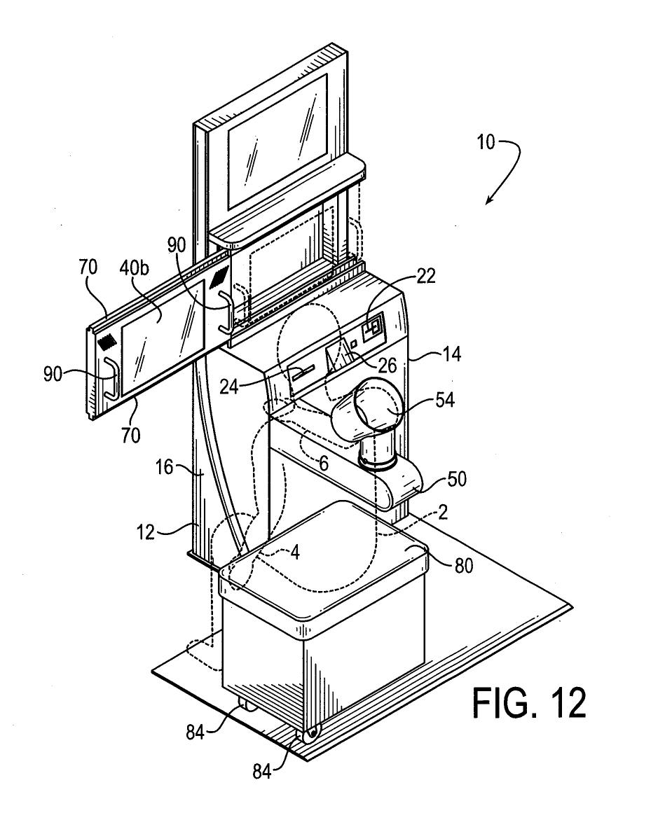

FIGS. 10 through 12 show three alternative embodiments for moving the display 40b. The display 40b includes handles 90 for moving the display 40b. In FIG. 10, the display 40b is attached to the kiosk 10 by arms 62 which pivot about pivot points 64. In FIG. 11, the display 40b is pivotally attached to the kiosk 10 at pivot point 66 and rotates in the direction of arrow 68. In FIG. 12, the display is 40b is slidably attached to the kiosk 10 by sliding rails 70.

These various embodiments allow the user 2 conveniently to place either his left arm 4 or right arm 6 into the blood pressure cuff 54 and to read the display 40b.

As will be apparent to those skilled in the art in light of the foregoing disclosure, many alterations and modifications are possible in the practice of this invention without departing from the spirit or scope thereof.

1. A blood pressure kiosk for measuring the blood pressure of a user, comprising:
a base having a right side and a left side;
a control unit mounted on the base;
a display mounted on the base for displaying information;
a seat near the base;
a support arm, the support arm supporting a blood pressure cuff, the support arm having two positions, a first position in which the support arm is near the right side of the base and a second position in which the support arm is near the left side of the base such that when the user sitting on the seat the user may put his left arm through the blood pressure cuff when the support arm is in the first position and when the user is sitting on the seat the user may put his right arm through the blood pressure cuff when the support arm is in the second position.

2. A blood pressure kiosk for measuring the blood pressure of a user, comprising:
a base having a right side and a left side;
a control unit mounted on the base;
a display mounted on the base for displaying information;
a seat near the base;
a support arm, the support arm including a blood pressure cuff, the blood pressure cuff having, two positions, a first position in which the blood pressure cuff is near the right side of the base and a second position in which the blood pressure cuff is near the left side of the base such that when the user is sitting on the seat the user may put his left arm through the blood pressure cuff when the blood pressure cuff is in the first position and when the user is sitting on the seat the user may put his right arm through the blood pressure cuff when the blood pressure cuff is in the second position.

3. The blood pressure kiosk of claim 1 wherein the support arm moves between the first position and the second position by sliding across the kiosk.

4. The blood pressure kiosk of claim 1 wherein the support arm moves between the first position and the second position by pivoting between the first position and the second position.

5. The blood pressure kiosk of claim 4 wherein the cuff is adapted for rotation about a vertical axis.

6. A blood pressure kiosk for measuring the blood pressure of a user, comprising:
a base having a right side and a left side;
a control unit mounted on the base;
a display mounted on the base for displaying information;
a seat near the base, the seat having two positions, a first position in which the seat is primarily between the left side of the base and right side of the base, and a second position in which the seat is laterally displaced with respect to the base;
a support arm, the support arm including a blood pressure cuff, the support arm located such that when the user is sitting on the seat in the first position, the user may put one of his arms through the blood pressure cuff and when the seat is in the second position, the user may put his other arm through the blood pressure cuff.

7. The blood pressure kiosk of claim 6 wherein the display is mounted such that the display has a first position in which the display is opposite the user when the user places one arm through the blood pressure cuff, and a second position in which the display is opposite the user when the user places his other arm through the blood pressure cuff.

8. The blood pressure kiosk of claim 7 further including a track on which the display is slidably mounted.

9. The blood pressure kiosk of claim 7 further including moving arms on which the display is mounted.

10. The blood pressure kiosk of claim 7 wherein the display moves between the first and second positions by rotation about a pivot.

11. The blood pressure kiosk of claim 6 wherein the seat moves from the first position to the second position by lateral movement.

12. The blood pressure kiosk of claim 6 wherein the seat moves from the first position to the second position by rotation about a vertical axis.
